(19) United States
(12) Patent Application Publication
(10) Pub. No.: US 2024/0074971 A1
(43) Pub. Date: Mar. 7, 2024

(54) NANO- IVERMECTIN COMPOSITIONS

(71) Applicants: Nefise Savrun, New York (US); Mehmet Ozgur Soydan, Kayseri (TR)

(72) Inventors: Nefise Savrun, New York (US); Mehmet Ozgur Soydan, Kayseri (TR)

(21) Appl. No.: 18/241,246

(22) Filed: Sep. 1, 2023

Related U.S. Application Data
(60) Provisional application No. 63/402,991, filed on Sep. 1, 2022.

(51) Int. Cl. A61K 9/00; A61K 9/10; A61K 31/7048; A61K 47/26; A61P 31/14

(52) U.S. Cl. CPC A61K 9/0078; A61K 9/10; A61K 31/7048; A61K 47/26; A61P 31/14

(57) ABSTRACT

A pharmaceutical composition of ivermectin nano-suspension is provided, which is suitable for nebulizer, where dispersion medium is water, containing at least 0.1 mg/mL tween 80 stabilizer.

### CROSS REFERENCE TO THE RELATED APPLICATIONS

This application claims priority to provisional application 63/402,991, filed on Sep. 1, 2022, the entire content of which is incorporated herein by reference.

### TECHNICAL FIELD

The present invention relates to a new nano-composition of ivermectin suitable for use as nebulization.

### BACKGROUND

During the COVID-19 pandemic, studies continued the effectiveness of many drugs to stop or slow the disease. One of these drugs, ivermectin, is currently approved by the FDA as antiparasitic drug and is on the WHO essential medicines list. Demonstration of the antiviral activity of ivermectin against SARS-CoV-2 in vitro accelerated the development studies for the use of the molecule in the treatment of COVID-19 [1]. For this purpose, many clinical studies have been initiated. When the clinical studies conducted for this purpose are examined, it is seen that in addition to the IV and oral administration of ivermectin, an inhaled way of use has been developed in order to reduce the side effects of the system and to act more intensely in the lung area damaged by the virus [2]. In these studies, it seems that ivermectin, which is prepared in the form of dry powder inhaler, is used. Since the use of dry powder inhalers requires minimum inspiratory flow for the patient to inhale the powder [3], it will not be possible to use this dosage form especially in patients who have difficulty in breathing, in children, and in unconscious patients.

When other studies aiming to develop nanoparticulate ivermectin, which were not carried out to transport ivermectin to the lungs, are examined, Ali et. al., nanoparticles were obtained by loading ivermectin into PLGA. It seems that the particle obtained here will not be suitable due to the limitation of inhalation of PLGA and it is actually a system that can provide controlled release[4]. Elmehy et. get. is a study focusing on the efficacy of niosomal ivermectin and nanocrystalline ivermectin after oral use. This study describes the preparation of nanocrystalline ivermectin by simply dissolving ivermectin (15 mg) in alcohol (3 mL) and adding it to water. It does not contain information such as nanocrystalline stability, suitability for use with nebulizer. All these parameters are the part that must be defined clearly and constitute the main critical step in the use of drugs by nebulization[5]. In another study, Aref et. get. developed a mucoadhesive nanosuspension containing ivermectin and brought it into nasal spray form to prevent the COVID-19-induced damage especially in the nasal region. In this study, poloxamer 407 and Poloxamer 188 were used to stabilize nanocrystals, and after the formation of nanocrystals, high-speed homogenization was performed to reduce aggregates. This formulation was then added to mucoadhesive polymer mixture (HPMC K15M (0.3% w/v), Carbopol 974P (0.1% w/v) and sodium alginate (0.2% w/v)). The study, which does not develop clarity on the issues such as the need for homogenization and post-nebulization stability of these nanocrystals obtained with Poloxamer, does not provide any solution to the problem of using ivermectin as inhalation with a nebulizer [6].

When other studies with ivermectin are examined, it is seen that composition development and preclinical studies suitable for use with nebulizer were carried out by Chaccour et al [7]. In this study, a composition was prepared by dissolving ivermectin, which is a water-insoluble drug, using ethanol co-solvent [7]. The main limitation of this approach is the possibility of causing irritation in the nose, throat and lungs after inhalation of ethanol [8]. Therefore, there is a need for a nebulized ivermectin composition that does not contain organic solvents as co-solvents. In preparing a solution for a water-insoluble drug, one of the most preferred composition is suspensions. “Ivermectin suspension composition” will make inhale use of ivermectin for patients having difficulty in breathing, children, and unconscious patients. Not being limited, preferred use of ivermectin is in treatment of COVID-19.

### SUMMARY

Although ivermectin has been shown to have antiviral activity against Sars-Cov-2 virus in in vitro studies, these studies have not yet been supported by appropriate clinical research results. Adverse effects, seen at high doses, were cited as the reason for failure. To overcome this problem, inhalation dosage form of ivermectin is being evaluated but developed composition have major limitation. Present invention is a stable, nano-sized suspension composition containing ivermectin particles, suitable for use as nebulization.

### DETAILED DESCRIPTION OF THE EMBODIMENTS

For this purpose, ivermectin suspension composition of present invention is formulated. By using nano-sized particles in suspension, the rate of dissolution in the lung tissue could be increased compared to conventional suspensions. This property makes the composition of invention suitable for use in nebulizers, and the property of hanging re-suspended particles for a long time without collapsing during use, is obtained.

### Example 1

The dispersion medium volume and the amount of ivermectin were determined according to the 1.2 mg/mL. This ivermectin concentration could be provide an opportunity to be filled directly into the vails in the manufacturing of final product. Accordingly, the first formulation tested is given in Table 1.

TABLE 1
Composition of Formulation F1

| Component | Function | Quantity (Per vial) | |
|---|---|---|---|
| Ivermectin | API | 6 | mg |
| Tween 80 | Stabilizer | 0.25 | mg |
| Acetone* | Organic Solvent | 1.5 | mL |
| Water | Dispersion Medium | 5 | mL |

*It is removed by evaporation and is not included in the final product.

The formulation given in Table 1 was prepared as follows: First, ivermectin was dissolved in the organic phase. The aqueous phase was prepared by dissolving Tween 80 in water. The organic phase was added dropwise to the continuously stirring water phase. The suspension obtained after evaporation of the acetone was evaluated. In the results of the measurements, 865 nm sized crystals were the majority, but 5 micrometer-sized aggregates were determined.

The inhalability of the prepared ivermectin suspension with a nebulizer was tested. For this, F1 were nebulized with the Mesilife NE-M01 Portable Mini Mesh Nebulizer and collected in a vial at the steam vent. For a short time, the nebulizer was clogged, which confirmed the resulting aggregates.

### Example 2

In order to solve the aggregation obtained in the first trial, the suitability of the nanosuspensions obtained by adding different amounts of stabilizer for use with nebulizer was tested. For this purpose, F2 was prepared.

TABLE 2
Composition of Formulation F2

| Component | Function | Quantity (Per vial) | |
|---|---|---|---|
| Ivermectin | API | 6 | mg |
| Tween 80 | Stabilizer | 0.5 | mg |
| Acetone* | Organic Solvent | 1.5 | mL |
| Water | Dispersion Medium | 5 | mL |

*It is removed by evaporation and is not included in the final product.

The nanosuspension was prepared and analyzed as described in Example 1. When the analysis results were examined, average particle size of nanocrystals were found as 663 nm and, F2 was easily nebulized with a nebulizer. It was determined that the average particle size of nanocrystals after nebulization increased over 1 micrometer.

### Example 3

Example 3 was tried by increasing the amount of tween 80 in order to solve the aggregation problem after nebulization.

TABLE 3
Composition of Formulation F3

| Component | Function | Quantity (Per vial) | |
|---|---|---|---|
| Ivermectin | API | 6 | mg |
| Tween 80 | Stabilizer | 0.5 | mg |
| Acetone* | Organic Solvent | 1.5 | mL |
| Water | Dispersion Medium | 5 | mL |

*It is removed by evaporation and is not included in the final product.

The nanosuspension was prepared as described in Example 1. The particle size was found to be about 470 nm. The inhalability of the prepared ivermectin solution with a nebulizer was tested. No aggregation was observed. Also the zeta potential was measured and found as −7 mV. Then, the particles were kept separately at room temperature and +4° C., and their stability was checked after a week. The size and zeta potential of the particles did not change in both ambient conditions, and no aggregation was observed.

### Example 4

In order to test whether it is possible to prepare the nanosuspension containing a much higher amount of ivermectin, the following example was tested.

TABLE 4
Composition of Formulation F4

| Component | Function | Quantity (Per vial) | |
|---|---|---|---|
| Ivermectin | API | 60 | mg |
| Tween 80 | Stabilizer | 5 | mg |
| Acetone* | Organic Solvent | 2.5 | mL |
| Water | Dispersion Medium | 5 | mL |

*It is removed by evaporation and is not included in the final product.

After the organic phase was dropped into the water phase, the suspension precipitated into large aggregates. Instead of preparing this more concentrated product, it was determined as a new stable nano-ivermectin suspension suitable for inhalation with the example 3 mesh nebulizer containing the ready-to-fill formulation as soon as the organic solvent is removed.

Pharmaceutical composition of the invention is a composition of ivermectin nano-suspension, suitable for nebulizer. The composition provide dispersion medium is water containing at least 0.1 mg/mL tween 80 stabilizer. Particle size of the nanosuspension is below 600 nm. Preferably amount of ivermectin is 1.2 mg/mL. The composition of invention may contain isotonicity agent or buffer. The composition is preferably used in treatment of lung diseases specially for treatment of Covid-19.

### REFERENCES

1. Caly, L., et al., The FDA-approved drug ivermectin inhibits the replication of SARS-CoV-2 in vitro.

Antiviral Research, 2020. 178: p. 104787.
2. ClinicalTrials.gov. Inhaled Ivermectin and COVID-19 (CCOVID-19). [cited 2022 May]; Available from: https://clinicaltrials.gov/ct2/show/NCT04681053?term=ivermectin&draw=4&rank=29.
3. Crompton, G. K., Dry powder inhalers: advantages and limitations. J Aerosol Med, 1991. 4(3): p. 151-6.
4. Ali, M., et al., Therapeutic efficacy of poly (lactic-co-glycolic acid) nanoparticles encapsulated ivermectin (nano-ivermectin) against brugian filariasis in experimental rodent model. Parasitology Research, 2014. 113(2): p. 681-691.
5. Elmehy, D. A., et al., Niosomal versus nano-crystalline ivermectin against different stages of Trichinella spiralis infection in mice. Parasitology Research, 2021. 120(7): p. 2641-2658.
6. Aref, Z. F., et al., Clinical, Biochemical and Molecular Evaluations of Ivermectin Mucoadhesive Nanosuspension Nasal Spray in Reducing Upper Respiratory Symptoms of Mild COVID-19. Int J Nanomedicine, 2021. 16: p. 4063-4072.
7. Chaccour, C., et al., Nebulized ivermectin for COVID-19 and other respiratory diseases, a proof of concept, dose-ranging study in rats. Scientific Reports, 2020. 10(1): p. 17073.
8. NJ-Health. Ethanol-Hazardous Substance Fact Sheet. [cited 2022 May]; Available from: https://www.nj.gov/health/eoh/rtkweb/documents/fx/0844.pdf.

What is claimed is:

1. A pharmaceutical composition of ivermectin nano-suspension, suitable for nebulizer, wherein dispersion medium is water, containing at least 0.1 mg/mL tween 80 stabilizer.

2. The pharmaceutical composition according to claim 1, wherein a particle size of the nano-suspension is below 600 nm.

3. The pharmaceutical composition according to claim 1, wherein an amount of ivermectin is 1.2 mg/mL.

4. The pharmaceutical composition according to claim 1, further comprising isotonicity agent and/or buffer.

5. The pharmaceutical composition according to claim 1, wherein the pharmaceutical composition is used in treatment of lung diseases.

6. The pharmaceutical composition according to claim 5, wherein the pharmaceutical composition is used in treatment of Covid-19.
